(19) United States
(12) Patent Application Publication
(10) Pub. No.: US 2025/0250545 A1
(43) Pub. Date: Aug. 7, 2025

(54) MSCS AND EXTRACELLULAR VESICLES

(71) Applicant: EXOSTEM BIOTEC LTD., Tel Aviv (IL)

(72) Inventor: Aharon BRODIE, Modiin (IL)

(73) Assignee: EXOSTEM BIOTEC LTD., Tel Aviv (IL)

(21) Appl. No.: 18/707,055

(22) PCT Filed: Nov. 4, 2021
(86) PCT No.: PCT/IL2021/051312

§ 371 Date: May 2, 2024

(51) Int. Cl. C12N 5/071; C12N 5/00

(52) U.S. Cl. CPC C12N 5/0697; C12N 5/0018; C12N 5/0075; C12N 2500/76; C12N 2500/84; C12N 2502/1305; C12N 2502/1311; C12N 2502/1335; C12N 2506/1346

(57) ABSTRACT

Methods of differentiating a mesenchymal stromal cell (MSC) to a first non-MSC cell fate, the method comprising contacting the MSC with extracellular vesicles (EVs), matrix-bound vesicles (MBVs) or a combination thereof are provided. Methods of producing artificial tissue by culturing MSCs with two sets of vesicles each comprising EVs, MBVs or both that differentiate MSCs to two different non-MSC cell fates, methods of culturing with reduced growth factors and method of differentiating an MSC to a muscle cell fate are also provided.

### CROSS REFERENCE TO RELATED APPLICATIONS

This application claims the benefit of priority of U.S. Provisional Patent Application No. 63/109,897, Nov. 5, 2020 titled “MSCS AND EXTRACELLULAR VESICLES”, the contents of which is incorporated herein by reference in its entirety.

### FIELD OF INVENTION

The present invention is in the field of stem cell culture and differentiation.

### BACKGROUND OF THE INVENTION

There is a great need for method of producing animal tissue, and not just animal cells, in culture. The ability to produce tissue, with a 3-dimensional structure similar to that of naturally occur tissue, is a burgeoning field. The therapeutic applications of artificially produced human tissue are nearly boundless and range from grafts to whole limb replacement in theory. The production of tissue from domesticated animal species is also highly sought.

There is an urgent need for meat alternatives due to increased demands for meat consumption and the fact that meat production is currently responsible for about 18% of greenhouse gas emissions, 8% of human water consumption and pollutes the water and air. The generation of cultured meat has the potential to replace animal-based meat and decrease the above-mentioned problems. In principle, cultured meat production is initiated by biopsy of adult cells/stem cells/progenitor cells. The isolated cells are expanded and then differentiated to myotubes and eventually to myocytes or adipocytes. Meat contains 90% muscle fibers, and the rest is fat and connective tissues. The optimal ratio of muscle to fat, the generation of extracellular matrix and scaffold proteins, and the assembly of all these cells results in edible cultured meat. Cultured meat textures are determined by a number of factors including the degree of muscle fiber maturation and alignment, the content of collagen and fat and the scaffold material and structure. Factors like the geometry of microcarriers in the bioreactor, and strain and nerve-like stimulation can affect muscle maturation and myotube formation.

There are a few limiting factors in the preparation of cultured meat, primary among them is the essential use of growth factors that promote muscle differentiation. Growth media containing growth factors represent the most expensive components in culture meat production. Due to the fact that the final product must be fit for human consumption, cheap alternatives are not available. Therefore, developing replacements for some or all of the growth factors is greatly needed. Even better would be a method that allows for recycling of the supplements added in place of the growth factors.

### SUMMARY OF THE INVENTION

The present invention provides methods of differentiating a mesenchymal stromal cell (MSC) to a first non-MSC cell fate, the method comprising contacting the MSC with extracellular vesicles (EVs), matrix-bound vesicles (MBVs) or a combination thereof. Methods of producing artificial tissue by culturing MSCs with two sets of vesicles each comprising EVs, MBVs or both that differentiate MSCs to two different non-MSC cell fates, methods of culturing with reduced growth factors and method of differentiating an MSC to a muscle cell fate are also provided.

According to a first aspect, there is provided a method of differentiating a mesenchymal stromal cell (MSC) to a first non-MSC cell fate, the method comprising contacting the MSC with purified or isolated extracellular vesicles (EVs), purified or isolated matrix-bound vesicles (MBVs) or a combination thereof derived from a second non-MSC source, or an MSC cell differentiated to the second non-MSC cell fate, thereby differentiating an MSC to a first non-MSC cell fate.

According to another aspect, there is provided a method of producing artificial tissue, the method comprising:
a. culturing a population of MSCs in a vessel;
b. adding to the vessel a first set of vesicles comprising purified or isolated EVs, purified or isolated MBVs or both, wherein the first set of vesicles is capable of differentiating an MSCs to a first non-MSC cell fate; and
c. adding to the vessel a second set of vesicles comprising purified or isolated EVs, purified or isolated MBVs or both, wherein the second set of vesicles is capable of differentiating an MSCs to a second non-MSC cell fate;
wherein the first and second non-MSC cell fates are different cell fates, thereby producing artificial tissue.

According to another aspect, there is provided a method of culturing a first cell in media, the method comprising contacting the media with isolated or purified EVs, MBVs or both from a second cell, wherein the media comprise reduced growth factor concentration as compared to a growth factor concentration required to culture the first cell in the absence of the isolated or purified EVs, MBVs or both.

According to another aspect, there is provided a method of culturing a first cell, the method comprising:
a. culturing the first cell in media for a time sufficient for the first cell to secrete vesicles;
b. removing a portion of the media;
c. concentrating, purifying or isolating secreted EVs, MBVs or both from the removed portion to produce a concentrated fraction; and
d. returning the concentrated fraction with new media to the culture comprising the first cell and continuing to culture the first cell;
thereby culturing a first cell.

According to another aspect, there is provided a method of differentiating a mesenchymal stromal cell (MSC) toward a muscle cell fate, the method comprising contacting the MSC with
a. an extracellular vesicle secreted by a satellite cell; and
b. an extracellular vesicle secreted by an MSC incubated with an HDAC inhibitor;
thereby differentiating the MSC toward a muscle cell fate.

According to another aspect, there is provided a method of trans-differentiating a non-MSC cell toward a muscle cell fate, the method comprising expressing MyoD in the non-MSC cell and contacting the non-MSC cell with at least one of:
a. a muscle cell;
b. an MSC differentiated toward a muscle cell fate;
c. an EV or MBV from a muscle cell; and
d. an EV or MBV from an MSC differentiated toward a muscle cell fate;
thereby differentiating the non-MSC cell toward a muscle cell fate.

According to some embodiments, the contacting comprises contacting with purified or isolated MBVs derived from a non-MSC source or MSC cell differentiated to a non-MSC fate.

According to some embodiments, the contacting comprises contacting with purified or isolated MBVs and purified or isolated EVs derived from a non-MSC source or MSC cell differentiated to a non-MSC fate.

According to some embodiments, the MBVs are adhered to a microcarrier, optionally wherein the microcarrier is an artificial scaffold.

According to some embodiments, the second non-MSC source is selected from a plant cell and from milk.

According to some embodiments, the plant cell is a soy cell or an oat cell.

According to some embodiments, the first and second non-MSC cell are from the same cell lineage.

According to some embodiments, the first and second non-MSC cell are the same cell type.

According to some embodiments, the non-MSC cell lineage is a muscle lineage, and wherein the first and second non-MSC cells are selected from a satellite cell, a myoblast and a myotubule.

According to some embodiments, the non-MSC cell lineage is an adipose lineage.

According to some embodiments, the non-MSC cell lineage is a bone lineage.

According to some embodiments, the isolated or purified EVs, MBVs or both are depleted of non-membrane enclosed growth factors.

According to some embodiments, the isolated or purified EVs, MBVs or both does not comprise conditioned media.

According to some embodiments, the MSCS are selected from adipose MSCs (AD-MSCs), bone marrow MSCs (BM-MSCs), umbilical cord MSCs (UC-MSCs), chorionic placenta MSCs (CH-MSCs), amniotic placenta MSCs (AM-MSCs) and dental pulp MSCs (DP-MSCs).

According to some embodiments, the first set of vesicles and the second set of vesicles are present in the vessel at the same time.

According to some embodiments, the first set of vesicles and the second set of vesicles are added simultaneously.

According to some embodiments, a first subpopulation of the population of MSCs differentiates to the first non-MSC cell fate and a second subpopulation of the population of MSCs differentiates to the second non-MSC cell fate.

According to some embodiments, the method further comprises culturing the population of MSCs with the first and second set of vesicles for a time sufficient for a first subpopulation of the population of MSCs to differentiate to the first non-MSC cell fate and a second subpopulation of the population of MSCs to differentiate to the second non-MSC cell fate.

According to some embodiments, the first non-MSC cell fate and the second non-MSC cell fate are selected from muscle cells, adipose cells and bone cells.

According to some embodiments, the artificial tissue is artificial meat and wherein the MSCs are non-human MSCs.

According to some embodiments, the non-human MSCs are selected from bovine MSCs, porcine MSCs, ovine MSCs, a hircine MSCs, fish MSCs and avian MSCs.

According to some embodiments, the continuing to culture the first cell comprises culturing with a reduced growth factor concentration as compared to a concentration of the growth factor required to culture the first cell in the absence of the extracellular vesicles.

According to some embodiments, the reduced growth factor concentration is devoid of the growth factor.

According to some embodiments, the second cell is a same cell type as the first cell.

According to some embodiments, the second cell is a different cell type as the first cell.

According to some embodiments, the first cell, the second cell or both is selected from a muscle cell, an adipose cell, a fibroblast, an MSC and an MSC differentiated toward a non-MSC cell fate.

According to some embodiments, the muscle cell is selected from a satellite cell and a myoblast.

According to some embodiments, culturing comprises increasing proliferation, decreasing senescence or both.

According to some embodiments, the first cell is an MSC and the method is a method of differentiating an MSC toward a non-MSC fate.

According to some embodiments, the non-MSC fate is selected from a muscle cell fate and an adipocyte cell fate.

According to some embodiments, the culturing is in a bioreactor.

According to some embodiments, the media is chemically defined media.

According to some embodiments, wherein the isolated or purified EVs, MBVs or both comprises MBVs.

According to some embodiments, the MBVs comprise a microcarrier coupled to the MBVs.

According to some embodiments, the microcarrier is an artificial scaffold, optionally wherein the microcarrier is biodegradable, edible or both.

According to some embodiments, the muscle cell is selected from a satellite cell, a myoblast and a myotube cell.

According to some embodiments, the muscle cell expresses MyoD.

According to some embodiments, the MSC incubated with an HDAC inhibitor is an MSC differentiated toward a muscle cell fate.

According to some embodiments, the HDAC inhibitor is Trichostatin A (TSA).

According to some embodiments, the contacting comprises culturing for a period of time sufficient for the differentiation.

According to some embodiments, the method further comprises confirming expression of a muscle cell marker in the differentiated MSC.

According to some embodiments, the muscle cell marker is MyoD.

According to some embodiments, the non-MSC cell is a terminally differentiated cell.

According to some embodiments, the non-MSC cell is a fibroblast.

According to some embodiments, the method comprises contacting the non-MSC cell with EVs from a satellite cell.

According to some embodiments, the extracellular vesicle comprises a diameter of between 30-150 nm.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-D: Bar graphs of (1A, 1B, 1C) MyoD reporter expression or (1C) MyHC reporter expression from MSCs cultured with (1A) extracellular vesicles from satellite cells, myotubules or fibroblasts, (1B-C) EVs combined with various growth factors, and (1D) EVs, MBVs or a combination thereof. *=P<0.001

FIGS. 2A-B: Bar graphs of MyoD reporter expression from MSCs cultured with (2A) extracellular vesicles from MSCs differentiated to a muscle fate, from satellite cells or a combination of the two and (2B) EVs and MBVs from MSCs differentiated to a muscle fate. *=P<0.001

FIGS. 3A-B: Bar graphs of adiponectin reporter expression from MSCs cultured with (3A) extracellular vesicles from adipocytes, satellite cells or fibroblasts and (3B) EVs and MBVs from adipocytes. Data from a MyoD reporter is also provided in 3B. **=P<0.001, *=P<0.01

FIG. 4: A bar graph of osteocalcin reporter expression from MSCs cultured with EVs, MBVs or a combination of both from osteoblasts. **=P<0.001, *=P<0.01

FIG. 5: A bar graph of relative MyOD mRNA expression from MSCs from three species cultured with EVs from muscle cells from that same species. *=P<0.001

FIG. 6: A bar graph of relative MyHC mRNA expression from human MSCs cultured with EVs from bovine milk, soy and oats. *=P<0.001

FIG. 7: A bar graph of relative MyHC mRNA expression from unmodified human fibroblasts or fibroblasts expressing MyoD cultured with or without EVs from human satellite cells. *=P<0.001

FIGS. 8A-B: Bar graphs of (8A) relative cell number and (8B) SA-beta-gal expression in MSCs cultured with EVs from various MSCs or Fibroblasts. **=P<0.001, *=P<0.01

FIG. 9: A bar graph of relative cell number of AD-MSCs cultured with EVs from various sources. *=P<0.001.

### DETAILED DESCRIPTION OF THE INVENTION

The present invention, in some embodiments, provides methods of differentiating MSCs. The present invention further concerns a method of culturing cells by adding extracellular vesicles (EVs) and matrix-bound vesicles (MBVs). Methods of producing an artificial tissue are also provided.

The present invention is based, in part, on the unexpected finding that MSCs can be fully differentiated to non-MSC fates using purified or isolated EVs and MBVs. Indeed, it was surprisingly found that these vesicles were superior to using cultured media as other molecules in the media appear to work against differentiation. It was further unexpectedly discovered that these vesicles contained sufficient survival inducing molecules as to allow for a reduction in the addition of growth factors to the culture. Growth factors are one of the most expensive parts of cell culture and especially so when scale in up for the growth of large numbers of cells or tissue. The ability to harvest EVs and MBVs from culture and recycle them to reduce the need for growth factors or serum greatly enables the scale up of cell culture. MBVs in particular were found to be highly useful for differentiation and sustaining culture, and surprisingly the combination of EVs and MBVs produced the strongest differentiation. Further, due to the extracellular matrix (ECM)-bound nature of these vesicles that can be easily provides to a culture or bioreactor adhered to microcarriers. The use of the microcarriers allows for localized growth and differentiation, that is that the cells that come in contact with the microcarriers and specifically effected by the MBVs while other cells are not. This allows for the addition of different sets of microcarriers with different MBVs attached, which in turn generate different differentiations within one vessel. Thus, a single population of MSCs can be differentiated to two or more non-MSC cell types as the same time. This allows, for the first time, the generation of multi-cell type tissue in a single vessel. Finally, it was discovered that EVs/MBVs not just from primary cells, but also from differentiated MSCs (differentiated toward the desired cell fate) could produce the desired differentiation. Even more surprising, the combination of EVs from primary cells and differentiated MSCs produced a synergistic increase in the differentiation observed.

### Methods of Differentiation

By a first aspect, there is provided a method of differentiating a mesenchymal stromal cell (MSC) toward a first non-MSC cell fate, the method comprising contacting said MSC with extracellular vesicles (EVs), matrix-bound vesicles (MBVs) or both, thereby differentiating an MSC to a first non-MSC cell fate.

By another aspect, there is provided a method of differentiating a mesenchymal stromal cell (MSC) toward a first non-MSC cell fate, the method comprising contacting said MSC with EVs, thereby differentiating an MSC to a first non-MSC cell fate.

By another aspect, there is provided a method of differentiating a mesenchymal stromal cell (MSC) toward a first non-MSC cell fate, the method comprising contacting said MSC with MBVs, thereby differentiating an MSC to a first non-MSC cell fate.

By another aspect, there is provided a method of differentiating a mesenchymal stromal cell (MSC) toward a first non-MSC cell fate, the method comprising contacting said MSC with EVs and MBVs, thereby differentiating an MSC to a first non-MSC cell fate.

By another aspect, there is provided a method of differentiating an MSC toward a muscle cell fate, the method comprising contacting the MSC with
a. a satellite cell or a satellite cell vesicle; and
b. an MSC differentiated toward a muscle cell fate or a vesicle of an MSC differentiated to a muscle cell fate;
   thereby differentiating the MSC toward a muscle cell fate.

By another aspect, there is provided a method of differentiating a non-MSC cell toward a muscle cell fate, the method comprising contacting the non-MSC cell
a. with an agent that induces myogenic differentiation in MSCs; and
b. with a muscle cell, an MSC differentiated toward a muscle cell fate or a vesicle therefrom;
   thereby differentiating a non-MSC cell toward a muscle cell fate.

By another aspect, there is provided a method of differentiating a non-MSC cell toward a muscle cell fate, the method comprising expressing MyoD in the non-MSC cell and contacting the non-MSC cell with a muscle cell, an MSC differentiated toward a muscle cell fate or a vesicle therefrom, thereby differentiating a non-MSC cell toward a muscle cell fate.

“MSC”, as used herein, refers to multipotent stromal stem cells having the ability to differentiate into osteoblasts, adipocytes, and chondroblasts as well as skeletal muscle and endothelial cells under some conditions. The term “multipotent” refers to stem cells which can give rise to many different cell types. MSCs are present in the bone marrow and adipose tissue, and are also present in peripheral blood, placenta, umbilical cord blood, dental pulp, among other tissues. In some embodiments, the MSC described herein originates and/or is isolated from amniotic placenta, chorionic placenta, umbilical cord, bone marrow, adipose tissue, amniotic fluid, or dental pulp. In some embodiments, an MSC is an undifferentiated MSC. Isolation of MSCs from these tissue sources is well known in the art and any method of isolation may be used. The markers, both cell surface and cytoplasmic, used for identifying these MSC population are also well known and can be found for example in International Patent Publication WO2018/083700, herein incorporated by reference in its entirety.

In some embodiments, MSC population is a population of MCSs having a unique profile, particularly an expression profile, including but not limited to, one or more unique proteins (e.g., surface markers and secreted proteins), genes, and one or more coding and non-coding RNAs (e.g., miRNA and lncRNA). In some embodiments, an MSC population is characterized by extracellular vesicles having a unique profile, secreted from said MSCs such as exosomes and extracellular vesicles. In some embodiments, an MSC population are MSCs all derived from one tissue source. In some embodiments, the MSCs are adipose MSCs (AD-MSCs). In some embodiments, the MSCs are bone marrow MSCs (BM-MSCs). In some embodiments, the MSCs are placental MSCs. In some embodiments, the MSCs are amniotic placenta MSCs (AM-MSCs). In some embodiments, the MSCs are chorionic placenta MSCs (CH-MSCs). In some embodiments, the MSCs are umbilical cord MSCs (UC-MSC). In some embodiments, the MSCs are dental pulp MSCs (DP-MSCs). In some embodiments, the MSCs are selected from CH-MSCs and UC-MSCs. In some embodiments, the MSCs are selected from CH-MSCs and AD-MSCs. In some embodiments, the MSCs are selected from CH-MSCs, UC-MSCs and AD-MSCs.

In some embodiments, the MSCs are human MSCs. In some embodiments, the MSCs are non-human MSCs. In some embodiments, the MSCs are derived from a non-human animal. In some embodiments, the animal is a mammal. In some embodiments, the animal is avian. In some embodiments, the animal is a fish. In some embodiments, the MSCs are bovine. In some embodiments, the MSCs are porcine. In some embodiments, the MSCs are murine. In some embodiments, the MSCs are canine.

A “population”, as used herein, refers to a cell culture wherein at least 50%, at least 55%, at least 60%, at least 65%, at least 70%, at least 75%, at least 80%, at least 85%, at least 90%, at least 95%, at least 99%, or all cells of the culture have a similar profile. Each possibility represents a separate embodiment of the invention. In some embodiments, a population is an enriched population. A “subpopulation”, as used herein, refers to an MSC populations being further manipulated by various growth conditions so as to endow a further unique expression profile to a subset of that population of MSCs. Each population consists of additional subpopulations exhibiting different combinations of expression profiles or different levels of expression. In some embodiments, a subpopulation is a differentiated population. In some embodiments, a subpopulation is a population of differentiated MSCs within a population of undifferentiated MSCs.

In some embodiments, the MSCs are in culture. In some embodiments, culture is tissue culture. In some embodiments, the MSCs are ex vivo. In some embodiments, the MSCs are in vitro. In some embodiments, the method is an in vitro method. In some embodiments, the method is an ex vivo method. In some embodiments, the method is a method of culturing. In some embodiments, contacting comprises adding the EVs, MBVs or both to the culture media. In some embodiments, the culture is a bioreactor. In some embodiments, the culture is a 2D culture. In some embodiments, the culture is a 3D culture.

“Extracellular vesicles”, and “EVs” as used herein, are interchangeable and refer to cell-derived extracellular vesicles secreted from various cells including but not limited to exosomes and microvesicles. EVs refer to free floating vesicles and not vesicles that are matrix or membrane bound. “Exosome”, as used herein, refers to cell-derived extracellular vesicles of endocytic origin that are secreted from cells. In some embodiments, an exosome comprises a diameter of 30-150 nm. In some embodiments, an exosome comprises a diameter of 30-100 nm. In some embodiments, an exosome comprises a diameter of 50-100 nm. In some embodiments, an exosome comprises a diameter of 50-150 nm. As a non-limiting embodiment, for the generation of exosomes cells are maintained with Opti-MEM and human serum albumin or 5% FBS that was depleted from exosomes. “Microvesicles”, as used herein, refers to cell-derived extracellular vesicles originating from the plasma membrane, with a size of 30-1000 nm, and secreted from MSCs. Methods of isolating and purifying EVs are well known in the art and are described hereinbelow. For example, after media is removed from cells various different types of EVs, or all EVs as a whole, can be isolated by differential centrifugation. In some embodiments, cells are grown in hypoxic conditions or incubated in medium with low pH so as to increase the yield of vesicles. In other embodiments, cells are exposed to radiation so as to increases vesicle secretion and yield. In some embodiments, the vesicle is EVs. In some embodiments, the vesicle is MBVs. In some embodiments, the vesicle is EVs and MBVs. In some embodiments, an EV is not an MBV. In some embodiments, an MBV is not an EV. In some embodiments, MBVs are a subtype of EVs. In some embodiments, the EVs are devoid of cells. In some embodiments, the EVs are depleted of cells. In some embodiments, cells are intact cells. In some embodiments, the EVs are isolated EVs. In some embodiments, the EVs are purified EVs. In some embodiments, the EVs are devoid of MBVs. In some embodiments, the EVs are depleted of MBVs. In some embodiments, the EVs are devoid of non-enclosed bounds growth factors. In some embodiments, the EVs are depleted of non-membrane enclosed growth factors.

“Matrix-bound vesicles” and “MBVs”, as used herein, are interchangeable and refer to extracellular vesicles that are secreted by various cells but are not soluble and free floating but rather bind to extracellular matrix (ECM) and are anchored there. The cargo of EVs and MBVs is known to be distinct and often unique. Methods of isolating and purifying MBVs are known in the art and are described hereinbelow. For example, media is removed from cells and cells are lightly disrupted and washed away. The MBVs can then be isolated from the ECM by enzymatic digestion of the ECM and differential centrifugation. Alternatively, the MBVs can be used still attached to the ECM or the ECM can be partially digested such that MBVs are still attached to fractions of ECM. In some embodiments, the MBVs are devoid of cells. In some embodiments, the MBVs are depleted of cells. In some embodiments, cells are intact cells. In some embodiments, the MBVs are isolated MBVs. In some embodiments, the MBVs are purified MBVs. In some embodiments, the MBVs are devoid of EVs. In some embodiments, the MBVs are depleted of EVs. In some embodiments, the MBVs are devoid of non-matrix bound EVs. In some embodiments, the MBVs are depleted of non-matrix bound EVs. In some embodiments, the MBVs are devoid of non-membrane enclosed growth factors. In some embodiments, the MBVs are depleted of non-membrane enclosed growth factors.

In some embodiments, depleted is at least 10, 15, 20, 25, 30, 35, 40, 45, 50, 55, 60, 65, 70, 75, 80, 85, 90, 92, 95, 97, 99 or 100% depleted. Each possibility represents a separate embodiment of the invention. In some embodiments, depleted is at least 70% depleted. In some embodiments, depleted is at least 80% depleted. In some embodiments, depleted is at least 90% depleted. In some embodiments, depleted is at least 95% depleted. In some embodiments, devoid is 100% depleted.

In some embodiments, MBVs are on a scaffold. In some embodiments, the scaffold is a solid scaffold. In some embodiments, the scaffold is an artificial scaffold. In some embodiments, the scaffold is a biodegradable scaffold. In some embodiments, the scaffold is a natural scaffold. In some embodiments, the scaffold is organic. In some embodiments, the scaffold is non-organic. In some embodiments, the scaffold is ECM. In some embodiments, the scaffold is a portion of ECM. In some embodiments, the scaffold is a fragment of ECM. In some embodiments, the scaffold is a collagen scaffold. In some embodiments, the scaffold is a bead. In some embodiments, the scaffold is a microcarrier. As used herein, the term “microcarrier” refers to support matrix or scaffold allowing for the growth of cells in a liquid culture. In some embodiments, a microcarrier comprises a sub-millimeter size. In some embodiments, a microcarrier comprises a sub-millimeter diameter. In some embodiments, a microcarrier comprises a diameter of between 50-900, 50-800, 50-700, 50-600, 50-500, 50-400, 50-300, 50-200, 50-100, 100-900, 100-800, 100-700, 100-600, 100-500, 100-400, 100-300, 100-200, 150-900, 150-800, 150-700, 150-600, 150-500, 150-400, 150-300, or 150-200 micrometers (um). Each possibility represents a separate embodiment of the invention. In some embodiments, a microcarrier comprises a diameter of between 100-300 μm. In some embodiments, the microcarrier is a bead. In some embodiments, the microcarrier is a polymer microcarrier.

Microcarriers further include, but are not limited to, cultured cell derived collagen or gelatin, hydrogenes containing animal tendons, eggshell membranes, or MSC-derived tendons, dextran alginate, cellulose, cytodex, cytopore 1 and 2, cytoline, and poly-e-carpolactone. In some embodiments, the microcarriers are edible.

In some embodiments, the scaffold comprises a surface configured to promote attachment. In some embodiments, the MBVs are attached to the scaffold. In some embodiments, the MBVs are adhered to the scaffold. In some embodiments, the MBVs are linked to the scaffold. In some embodiments, the MBVs are conjugated to the scaffold. In some embodiments, the attachment is linking. In some embodiments, the linking is chemical linking. In some embodiments, the linking is covalent linking. In some embodiments, attachment is physical integration. In some embodiments, the attachment is printing on the carrier/scaffold. In some embodiments, the scaffold is printed. In some embodiments, the scaffold and MBVs are printed.

“Extracellular matrix”, and “ECM”, as used herein, are interchangeable and refer to the extracellular molecules secreted by the MSCs which provide a structural and biochemical support to surrounding cells. In some embodiments, the ECM comprises membranes. In some embodiments, the ECM is structured. In some embodiments, the ECM comprises collagen. In some embodiments, the ECM comprises a structured collagen fiber network.

“Conditioned media”, as used herein, refers to media in which a cell population has been growing. Conditioned media generally comprises all soluble secreted cellular components. Unless specific care has been taken to disrupt the ECM, conditioned media will not contain MBVs. Indeed, the standard use of the term “conditioned media” does not include ECM or MBVs. In some embodiments, the conditioned media comprises the extracellular vesicles secreted by cells. In some embodiments, the conditioned media comprises proteins secreted by cells. In some embodiments, the conditioned media comprises the secretome of the cells. As used herein, the term “secretome”, refers to any soluble substance secreted by a cell. In some embodiments, a secretome comprises any or all of secreted proteins, secreted nucleic acid molecules, and secreted soluble vesicles.

As used herein, the term “non-MSC” and “non-MSC cell fate” are used interchangeably and refer for any cell type or state other than undifferentiated MSCs. In some embodiments, the non-MSC cell fate is a cell type to which MSCs naturally differentiate. In some embodiments, the non-MSC cell fate is a cell type to which MSCs do not naturally differentiate. In some embodiments, the non-MSC cell fate is a cell fate that can be reached by trans-differentiation of an MSC. In some embodiments, the non-MSC is a cell of the adipose lineage. In some embodiments, the non-MSC is a cell of the bone lineage. In some embodiments, the non-MSC is a cell of the muscle lineage. In some embodiments, a muscle cell is selected from a satellite cell, a myoblast and a myotubule. In some embodiments, a muscle cell is a satellite cell. In some embodiments, a muscle cell is a myoblast. In some embodiments, a muscle cell is a myotubule. In some embodiments, an adipose cell is an adipocyte. In some embodiments, a bone cell is selected from an osteoblast, an osteoclast, an osteocyte and a bone lining cell. In some embodiments, a bone cell is an osteoblast. In some embodiments, a bone cell is an osteoclast. In some embodiments, a bone cell is an osteocyte. In some embodiments, a bone cell is a bone lining cell. In some embodiments, a non-MSC is a cell of a connective tissue lineage. In some embodiments, a connective tissue cell is a fibroblast. In some embodiments, a connective tissue cell is a fibrocyte. In some embodiments, a connective tissue cell is a tenocyte. In some embodiments, a connective tissue cell is a stromal cell. In some embodiments, a connective tissue cell is a pericyte. In some embodiments, the connective tissue is a tendon. In some embodiments, the connective tissue is a ligament. In some embodiment, a non-MSC is a cell of a tendon lineage. In some embodiments, a tendon cell is a tenocyte. In some embodiments, a non-MSC is a cell of the cartilage lineage. In some embodiments, a cartilage cell is a chondrocyte. In some embodiments, a non-MSC is a cell of a neuronal lineage. In some embodiments, a neuronal cell is a neuron. In some embodiments, a neuronal cell is a differentiated neuron. In some embodiments, a neuronal cell is a neuronal progenitor cell. In some embodiments, a neuronal cell is a neuronal precursor cell. In some embodiments, a neuronal cell is an astrocyte. In some embodiments, a neuronal cell is a motor neuron. In some embodiments, a non-MSC is a cell from a lineage selected from muscle, adipose, bone, connective tissue, and neuron.

In some embodiments, the MSCs are contacted with EVs. In some embodiments, the MSCs are contacted with MBVs. In some embodiments, the MSCs are contacted with EVs and MBVs. In some embodiments, the MSCs are contacted with a combination of EVs and MBVs. In some embodiments, EVs, MBVs or both do not comprise conditioned media. In some embodiments, EVs do not comprise soluble proteins. In some embodiments, EVs do not comprise non-membrane enclosed. In some embodiments, EVs do not comprise soluble and/or non-membrane enclosed proteins. In some embodiments, MBVs do not comprise soluble proteins. In some embodiments, MBVs do not comprise non-membrane enclosed proteins. In some embodiments, MBVs do not comprise soluble and/or non-membrane enclosed proteins. In some embodiments, the EVs, MBVs or both are washed to remove soluble proteins adhered to the surface of the vesicles.

In some embodiments, the EVs are enriched EVs. In some embodiments, the EVs are isolated EVs. In some embodiments, the EVs are purified EVs. In some embodiments, the EVs are essentially pure. In some embodiments, the EVs are substantially pure. In some embodiments, the EVs are devoid of soluble, non-membrane enclosed proteins. In some embodiments, the EVs are washed. In some embodiments, the EVs are devoid of soluble, non-membrane enclosed growth factors. In some embodiments, the proteins are growth factors. In some embodiments, the EVs are devoid of cells. In some embodiments, cells are intact cells. In some embodiments, the EVs are devoid of MBVs. In some embodiments, the EVs are concentrated.

In some embodiments, the MBVs are enriched MBVs. In some embodiments, the MBVs are isolated MBVs. In some embodiments, the MBVs are purified MBVs. In some embodiments, the MBVs are essentially pure. In some embodiments, the MBVs are substantially pure. In some embodiments, the MBVs are devoid of soluble, non-membrane enclosed proteins. In some embodiments, the MBVs are washed. In some embodiments, the MBVs are devoid of soluble, non-membrane enclosed growth factors. In some embodiments, the proteins are growth factors. In some embodiments, the MBVs are devoid of cells. In some embodiments, cells are intact cells. In some embodiments, the MBVs are devoid of EVs. In some embodiments, the MBVs are devoid of ECM. In some embodiments, the MBVs are depleted of ECM. In some embodiments, devoid is substantially devoid. In some embodiments, the MBVs are concentrated.

As used herein the term “separating”, “excluding” or “isolating” is intended to mean that the material has been completely, substantially or partially separated, isolated, excluded or purified from other components. As used herein, the term “isolated” refers to vesicles that are essentially free or removed from contaminating cellular and/or culture components, such as carbohydrates, lipids, proteins or other impurities associated with the vesicles in nature or culture. In some embodiments, isolated is isolated by differential centrifugation. Methods of preparing EVs and MBVs will, in most cases isolate and/or purify them. Added steps, such as washing, centrifuging or filtering can be done to further increase the purity. In some embodiments, purified comprises at least 70% purity. In some embodiments, purified comprises at least 75% purity. In some embodiments, purified comprises at least 80% purity. In some embodiments, purified comprises at least 85% purity. In some embodiments, purified comprises at least 90% purity. In some embodiments, purified comprises at least 95% purity. In some embodiments, purified comprises at least 97% purity. In some embodiments, purified comprises at least 99% purity. In some embodiments, purified comprises 100% purity.

In some embodiments, the enriched EVs comprises at least 50%, 60%, 70%, 80%, 90%, 95%, 97%, 99% or 100% EVs. Each possibility represents a separate embodiment of the invention. In some embodiments, the enriched MBVs comprises at least 50%, 60%, 70%, 80%, 90%, 95%, 97%, 99% or 100% EVs. Each possibility represents a separate embodiment of the invention. As used herein, “substantially devoid” refers to having only a very small contamination of undesired components. In some embodiments, a substantially pure population has less than 10%, 9%, 8%, 7%, 6%, 5%, 4%, 3%, 2%, 1%, 0.5%. 0.1%, 0.05% or 0.01% contaminating components. Each possibility represents a separate embodiment of the invention.

In some embodiments, the method further comprises providing the MSC. In some embodiments, the providing comprises selecting an MSC population from a mixture of cells, a tissue or an organ. In some embodiments, the selecting comprises measuring expression of at least one surface marker on the surface of a cell of the mixture of cells, tissue or organ and selecting a cell expressing at least one MSC surface marker. In some embodiments, an MSC surface marker is selected from CD73, CD105, CD90, CD146, and CD44 expression and absence of MHCII expression. In some embodiments, an MSC surface marker is selected from CD10, CD13, CD29, CD1, CD73, CD105, CD90, CD146, and CD44 expression and absence of MHCII expression, absence of CD14, absence of CD34, and absence of CD45. In some embodiments, an MSC surface marker is selected from CD9, CD10, CD13, CD26, CD29, CD44, CD36, CD46, CD47, CD49a, CD49b, CD49c, CD49d, CD49e, CD50, CD51/61, CD54, CD55, CD58, CD59, CD61, CD63, CD71, CD73, CD81, CD83, CD87, CD90, CD91, CD95, CD97, CD98, CD99, CD105, CD108, CD109, CD140b, CD142, CD146, CD147, CD151, CD164, CD165, CD166, CD273, β2-microglobulin, HLA-A, B, C, HLA-A2, and STRO1. In some embodiments, isolating MSCs comprises isolation of cells expressing a plurality of MSC surface markers.

In some embodiments, the vesicles are derived from a non-MSC source. In some embodiments, the vesicles are non-MSC vesicles. In some embodiments, the vesicles are derived from a second non-MSC source. In some embodiments, a source is a cell. In some embodiments, the non-MSC source is a plant cell. In some embodiments, the plant is soy. In some embodiments, soy is soybean. In some embodiments, soy is Glycine max. In some embodiments, the plant is a legume. In some embodiments, the plant is oats. In some embodiments, oats are Avena sativa. In some embodiments, the plant is a grass. In some embodiments, the plant is a grain. In some embodiments, the grain is a cereal grain. In some embodiments, the plant is a vegetable. In some embodiments, the plant is a fruit. In some embodiments, the plant is a legume. In some embodiments, the plant is quinoa. In some embodiments, the plant is high in protein. In some embodiments, the plant is high in amino acids. In some embodiments, the plant is high in starch. In some embodiments, the plant is agricultural crop. In some embodiments, the plant is cannabis. In some embodiments, the plant is not cannabis. In some embodiments, the vesicle does not comprise Genistein. In some embodiments, the non-MSC source is a fungal cell. In some embodiments, the fungus is a mushroom. In some embodiments, the method does not comprise contacting the MSC with soluble, non-membrane enclosed Genistein. In some embodiments, the non-MSC source is milk. In some embodiments, the milk is human milk. In some embodiments, the milk is non-human milk. In some embodiments, the milk is bovine milk. In some embodiments, the milk is from a domesticated mammal. In some embodiments, the second non-MSC source is a cell type to which an MSC can be differentiated. In some embodiments, the second non-MSC source is a cell type to which an MSC can be transdifferentiated.

As used here, “differentiated” refers to a process by which a less specialized cell becomes a more specialized cell. As used herein, “transdifferentiate” refers to a non-natural process by which a less specialized cell becomes a more specialized cell which it does not differentiate to in nature. Thus, it will be understood by a skilled artisan that differentiation refers to any type of differentiation that can occur whether it occurs in nature or only though a manmade process; while transdifferentiate refers to only differentiations that do not occur in nature and require a man-made induction to make the cell transdifferentiate.

In some embodiments, a non-human animal is any animal species other than humans. In some embodiments, a non-human animal is a veterinary animal. In some embodiments, a non-human animal is a domesticated animal. In some embodiments, a non-human animal is a farm animal. In some embodiments, a non-human animal is a captive-bred animal. In some embodiments, a non-human animal is an animal bred for food production. In some embodiments, the non-human animal is selected from cow, sheep, pig, goat, fish and fowl. In some embodiments, the non-human animal is selected from cow, sheep, pig, goat, dog, mouse, fish and fowl. In some embodiments, the non-human animal is bovine. In some embodiments, bovine is cow. In some embodiments, bovine is buffalo. In some embodiments, the non-human animal is ovine. In some embodiments, the non-human animal is porcine. In some embodiments, the non-human animal is hircine. In some embodiments, the non-human animal is canine. In some embodiments, the non-human animal is murine. In some embodiments, the non-human animal is avian. Examples of fowl, and in particular fowl used for consumption are well known in the art and include, but are not limited to, chicken, turkey, duck, pheasant, goose and quail. In some embodiments, the non-human animal is a fish. In some embodiments, the MSCs are selected from human MSCs, bovine MSCs, porcine MSCs, ovine MSCs, hircine MSCs, canine MSCs, murine MSCs, fish MSCs and avian MSCs. In some embodiments, the MSCs are selected from bovine MSCs, porcine MSCs, ovine MSCs, hircine MSCs, canine MSCs, murine MSCs and avian MSCs. In some embodiments, the MSCs are selected from human MSCs, porcine MSCs, canine MSCs and murine MSCs. In some embodiments, the vesicles are selected from human vesicles, bovine vesicles, porcine vesicles, ovine vesicles, hircine vesicles, canine vesicles, murine vesicles, fish vesicles and avian vesicles. In some embodiments, the vesicles are selected from bovine vesicles, porcine vesicles, ovine vesicles, hircine vesicles, canine vesicles, murine vesicles, fish vesicles and avian vesicles. In some embodiments, the MSCs and second non-MSC source are from the same species. In some embodiments, the MSCs and second non-MSC source are from different species.

In some embodiments, the first and second non-MSC cells are from the same cell lineage. In some embodiments, the first and second non-MSC cells are the same cell type. In some embodiments, the first and second non-MSC cells are from different cell lineages. In some embodiments, the first and second non-MSC cells are from the same cell lineage but are different cell types. In some embodiments, the first non-MSC cell is selected from a satellite cell, a myoblast and a myotubule and the second non-MSC is a muscle cell. In some embodiments, the first non-MSC cell is a satellite cell and the second non-MSC is a muscle cell. In some embodiments, the first non-MSC cell is a myoblast and the second non-MSC is a muscle cell. In some embodiments, the first non-MSC cell is a myotubule and the second non-MSC is a muscle cell. In some embodiments, the first non-MSC cell is a muscle cell and the second non-MSC is a muscle cell. In some embodiments, the first non-MSC is an adipocyte and the second non-MSC is an adipose cell. In some embodiments, the first non-MSC is a bone cell and the second non-MSC is a bone cell. In some embodiments, the first non-MSC cell fate, the second non-MSC cell fate or both are selected from muscle cells, adipose cells and bone cells. In some embodiments, the first non-MSC cell fate, the second non-MSC cell fate or both are selected from muscle cells, and adipose cells.

In some embodiments, the second non-MSC cell is an MSC differentiated to the second non-MSC cell. In some embodiments, the second non-MSC cell is an MSC that has been differentiated to a non-MSC cell. In some embodiments, the MSC was differentiated before the vesicles were derived from the cell. In some embodiments, the second non-MSC is selected from an MSC differentiated to a muscle cell, an MSC differentiated to an adipose cell and an MSC differentiated to a bone cell. In some embodiments, the second non-MSC is selected from an MSC differentiated to a muscle cell, and an MSC differentiated to an adipose cell. In some embodiments, the second non-MSC is an MSC differentiated into a muscle cell. In some embodiments, the second non-MSC is an MSC differentiated into an adipose cell. In some embodiments, the second non-MSC is an MSC differentiated into a bone cell.

In some embodiments, toward is into. In some embodiments, a cell fate is the cell. In some embodiments, toward a muscle cell fate is to a muscle cell fate. In some embodiments, toward a muscle cell fate is into a muscle cell. In some embodiments, the method is a method of partial differentiation. In some embodiments, the method is a method of complete differentiation. In some embodiments, a completely differentiated MSC is terminally differentiated. In some embodiments, a completely differentiated MSC is no longer immunosuppressive. In some embodiments, a completely differentiated MSC expresses MHC class II surface molecules. In some embodiments, a muscle cell expresses myoblast determination protein 1 (MyoD). In some embodiments, a muscle cell is characterized by expression of MyoD. In some embodiments, the MSC is differentiated into a cell expressing MyoD. In some embodiments, the method further comprises detecting MyoD expression in the differentiated MSC. In some embodiments, the method further comprises detecting expression of a muscle cell marker in the differentiated cell. In some embodiments, the method further comprises confirming expression of a muscle cell marker in the differentiated cell. In some embodiments, the muscle cell marker is MyoD. In some embodiments, the muscle cell marker is selected from MyoD, Myf6, Myf5, MRF4, ITGA7, osteoprotegerin, Irisin, dystrophin, Myosin heavy chain, myogenin, PAX7, TALNEC2, C-MET, G-CSF, osteoprotegerin, IL-10, and MEF2A expression and absence of osteocalacin, PPARG3, and COL2A1 expression.

The MSC differentiated into a muscle cell may be differentiated by any method known in the art. Numerous methods of performing this differentiation are known and several are provided hereinbelow in the Methods section, further a method may be selected from the methods presented in International Patent Publication WO2017/199250 the contents of which are hereby incorporated by reference in their entirety. In some embodiments, an MSC differentiated into a muscle cell is an MSC contacted with an HDAC inhibitor. In some embodiments, contacted comprises incubated. In some embodiments, the HDAC inhibitor is Trichostatin A (TSA). In some embodiments, an MSC differentiated into a muscle cell is an MSC contacted with 5-Aza-2′-deoxycytidine (5-aza). In some embodiments, an MSC differentiated into a muscle cell is an MSC contacted with a Wnt activator. In some embodiments, an MSC differentiated into a muscle cell is an MSC contacted with a sonic hedgehog (Shh) inhibitor. In some embodiments, an MSC differentiated into a muscle cell is an MSC contacted with a myostatin inhibitor.

In some embodiments, the differentiation further comprises a standard method of differentiation. It will be understood by a skilled artisan that by combining EV/MBV based differentiation with a standard differentiation the combined effect can be greater. In some embodiments, a standard method of differentiation is a method provided hereinbelow. In some embodiments, a standard method of myogenic differentiation is expression of MyoD. In some embodiments, expression is forced expression. In some embodiments, expression is overexpression. In some embodiments, expression is ectopic expression.

The MSC differentiated into an adipose cell or bone cell may be differentiated by any method known in the art. Numerous methods of performing this differentiation are known and for example a method of adipogenic differentiation may be selected from the methods provided in Scott et al., 2011, “Current methods of adipogenic differentiation of mesenchymal stem cells”, Stem cells Dev., October; 20 (10): 1793-804 and in International Patent Publication WO2013/184966 the contents of which are hereby incorporated by reference in their entirety. Similarly, a method of osteogenic differentiation may be selected for example from the methods provided in Ciuffreda et al., 2016, “Protocols for in vitro differentiation of human mesenchymal stem cells in osteogenic chondrogenic and adipogenic lineages”, Methods Mol. Biol., 1416:149-158 and in European Patent Publication EP2899266 the contents of which are hereby incorporated by reference in their entirety.

In some embodiments, contacting comprises incubating. In some embodiments, contacting comprises culturing. In some embodiments, contacting is for a period of time sufficient for the differentiation. In some embodiments, contacting is for a period of time sufficient to start the differentiation. In some embodiments, contacting is for a period of time sufficient to finish the differentiation. In some embodiments, the period of time is at least 1, 2, 3, 4, 5, 6, 7, 8, 9, 10, 11, 12, 13 or 14 days. Each possibility represents a separate embodiment of the invention. In some embodiments, the period of time is at least 1 day. In some embodiments, the period of time is at least 2 days. In some embodiments, the period of time is at least 3 days. In some embodiments, the period of time is at least 5 days. In some embodiments, the period of time is between 1-14, 1-10, 1-7, 1-5, 1-3, 2-14, 2-10, 2-7, 2-5, 2-3, 3-14, 3-10, 3-7 and 3-5 days. Each possibility represents a separate embodiment of the invention.

In some embodiments, the contacting is in the presence of growth factors. In some embodiments, the culturing is in the presence of growth factors. In some embodiments, in the presence of growth factors is in media supplemented with growth factors. Example of growth factors include, but are not limited to insulin-like growth factor (IGF), hepatocyte growth factor (HGF), platelet-derived growth factor (PDGF), brain-derived growth factor (BDNF), fibroblast growth factor (FGF) and noggin. In some embodiments, IGF is IGF-1. In some embodiments, FGF is FGF1. In some embodiments, FGF is FGF2. In some embodiments, the growth factor is IGF. In some embodiments, the growth factor is HGF. In some embodiments, the growth factor is PDGF. In some embodiments, the growth factor is IGF, HGF and PDGF. In some embodiments, the growth factor is selected from IGF, HGF and PDGF. In some embodiments, the growth factor is selected from BDNF, noggin, HGF, PDGF, FGF and IGF.

In some embodiments, the non-MSC cell is not a stem cell. In some embodiments, the non-MSC cell is a terminally differentiated cell. In some embodiments, the non-MSC cell is a cell that does not naturally differentiated to a muscle cell fate. In some embodiments, a muscle cell fate is a cell of a muscle lineage. In some embodiments, the non-MSC cell is a connective tissue cell. In some embodiments, the non-MSC cell is a fibroblast. In some embodiments, differentiation of a non-MSC cell is trans-differentiation. In some embodiments, the differentiation is trans-differentiation. As used herein, the term “trans-differentiation” refers to the differentiation of a cell to a cell type to which it does not naturally differentiate. Thus, trans-differentiation is a non-natural process.

In some embodiments, an agent that induces myogenic differentiation in an MSC is an agent recited hereinbelow for myogenic differentiation. In some embodiments, the agent is an agent selected from International Patent Publication WO2017/199250 the contents of which are hereby incorporated by reference in their entirety. In some embodiments, the agent is 5′aza. In some embodiments, the agent is TSA. In some embodiments, the agent is a Wnt activator. In some embodiments, the agent is a Shh inhibitor. In some embodiments, the agent is a TGF-beta 1 receptor inhibitor. In some embodiments, the agent is a MyoD expression vector. In some embodiments, the agent is MyoD protein. In some embodiments, contacting with the agent is expressing MyoD in the non-MSC cell. In some embodiments, expressing is forced expression. In some embodiments, expression is ectopic expression. In some embodiments, expression is over expression. In some embodiments, the method comprises expressing MyoD in the non-MSC cell.

In some embodiments, the non-MSC cell is contacted with a muscle cell. In some embodiments, the muscle cell is a satellite cell. In some embodiments, the non-MSC cell is contacted with a muscle cell vesicle. In some embodiments, the vesicle is an EV. In some embodiments, the vesicle is an MBV. In some embodiments, the non-MSC cell is contacted with an MSC differentiated toward a muscle cell fate. In some embodiments, the non-MSC cell is contacted with a vesicle of a MSC differentiated toward a muscle cell fate. In some embodiments, the vesicle is an EV. In some embodiments, the vesicle is an MBV.

### Methods of Tissue Generation

By another aspect, there is provided a method of producing tissue, the method comprising contacting a plurality of MSCs with a first set of vesicles capable of differentiating an MSC toward a first non-MSC cell and a second set of vesicles capable of differentiating an MSC toward a second non-MSC cell fate, thereby producing tissue.

In some embodiments, the tissue is artificial tissue. In some embodiments, the tissue is man-made tissue. In some embodiments, the tissue comprises cellular organization similar to natural tissue. In some embodiments, the tissue is artificial meat. In some embodiments, the tissue is edible tissue. In some embodiments, the tissue is human tissue. In some embodiments, the tissue is replacement tissue. In some embodiments, the tissue is non-human tissue. In some embodiments, the tissue is chemically defined. In some embodiments, the tissue is devoid of inedible components.

In some embodiments, the plurality of MSC is a population of MSCs. In some embodiments, a plurality is at least 2 MSCs. In some embodiments, the plurality is at least 100, 500, 1000, 5000, 10000, 50000, 100000, 500000, 1000000, 5000000, 10000000, 50000000, or 100000000 MSCs. Each possibility represents a separate embodiment of the invention.

In some embodiments, the plurality of MSCs are in culture. In some embodiments, culture is tissue culture. In some embodiments, culture comprises incubation in culture medium. In some embodiments, the medium is a chemically defined medium. As used herein, the term “chemically defined media” refers to a medium in which all the chemical components are known. In some embodiments, chemically defined media is devoid of animal-based products. In some embodiments, chemically defined media is devoid of animal-based proteins. In some embodiments, the media is devoid of human components. In some embodiments, the media is devoid of human proteins. In some embodiments, the media is devoid of inedible components. In some embodiments, the media is growth media. In some embodiments, the media is MSC media. In some embodiments, the media is differentiation media. In some embodiments, the media is not differentiation media. In some embodiments, the media is muscle cell media. In some embodiments, the media is OptiMeM.

In some embodiments, the MSCs are cultured in a vessel. Vessels for tissue culture are well known in the art and any such container may be used, including, but not limited to tissue culture plates, flasks, dishes, and reactors. It will be understood that the method of producing tissue is performed in one container, i.e., a single population of MSCs is differentiated to two different non-MSC populations in the same vessel. Thus, it is not a method where the differentiations are carried out separately and the cells are then mixed together after the differentiation.

In some embodiments, the method comprises adding the first set of vesicles to the plurality of MSCs. In some embodiments, the adding is to the culture. In some embodiments, the adding is to the vessel. In some embodiments, the vessel is a container. In some embodiments, the method comprises adding the second set of vesicles to the plurality of MSCs. In some embodiments, the first and second sets are added sequentially. In some embodiments, the first and second sets are added simultaneously. In some embodiments, sequentially comprises an interval of time that is shorter than the time required for the MSCs to differentiate. In some embodiments, the first and second sets are present in the vessel at the same time. In some embodiments, the first and second sets are present in the culture at the same time.

In some embodiments, the first set of vesicles, the second set of vesicles or both comprises EVs. In some embodiments, the first set of vesicles, the second set of vesicles or both consist of EVs. In some embodiments, the first set of vesicles, the second set of vesicles or both comprises MBVs. In some embodiments, the first set of vesicles, the second set of vesicles or both consist of MB In some embodiments, the first set of vesicles, the second set of vesicles or both comprises EVs and MBVs. In some embodiments, the first set of vesicles, the second set of vesicles or both consist of MBVs, and EVs. In some embodiments, the first set of vesicles, the second set of vesicles or both comprises EVs, MBVs or both. In some embodiments, the first set of vesicles, the second set of vesicles or both consist of MBVs, EVs, or both. In some embodiments, the vesicles are purified. In some embodiments, the vesicles are isolated. In some embodiments, the vesicles are concentrated.

In some embodiments, the first set is capable of differentiating an MSC toward a first non-MSC cell fate. In some embodiments, the first set is configured to differentiate an MSC toward a first non-MSC cell fate. In some embodiments, the first set differentiates MSCs toward a first non-MSC cell fate. In some embodiments, the first set is when contacted with an MSC induces differentiation of the MSC to a first non-MSC cell fate. In some embodiments, the first set comprises vesicles from the first non-MSC cell fate. In some embodiments, the first set comprises vesicles from an MSC differentiated to the first non-MSC cell fate.

In some embodiments, the second set is capable of differentiating an MSC toward a second non-MSC cell fate. In some embodiments, the second set is configured to differentiate an MSC toward a second non-MSC cell fate. In some embodiments, the second set differentiates MSCs toward a second non-MSC cell fate. In some embodiments, the second set is when contacted with an MSC induces differentiation of the MSC to a second non-MSC cell fate. In some embodiments, the second set comprises vesicles from the second non-MSC cell fate. In some embodiments, the second set comprises vesicles from an MSC differentiated to the second non-MSC cell fate. In some embodiments, the first and second non-MSC cell fates are different cell fates. In some embodiments, the first and second non-MSC cell fates are different cell types.

In some embodiments, a first subpopulation of the plurality of MSCs differentiates to the first non-MSC cell fate. In some embodiments, a second subpopulation of the plurality of MSCs differentiates to the second non-MSC cell fate. In some embodiments, the tissue comprises at least two cell types. In some embodiments, the tissue comprises at least two cell fates. In some embodiments, the first and second non-MSC cell fates are from different lineages. In some embodiments, the tissue comprises at least two different cell lineages. In some embodiments, the tissue comprises cells of at least two lineages.

In some embodiments, the method further comprises culturing the plurality of MSCs with the first and second sets for a time sufficient for differentiation. In some embodiments, the time is a time sufficient for a first subpopulation of the plurality of MSCs to differentiation toward the first non-MSC cell fate. In some embodiments, the time is a time sufficient for a second subpopulation of the plurality of MSCs to differentiation toward the second non-MSC cell fate. In some embodiments, a sufficient time is at least 5 days. In some embodiments, a sufficient time is at least 1 week. In some embodiments, a sufficient time is at least 2 weeks.

In some embodiments, the MBVs are on scaffolds. In some embodiments, the tissue comprises the scaffold. In some embodiments, the scaffolds are organic. In some embodiments, the scaffolds are biodegradable the method comprises culturing for a time sufficient for the scaffolds to degrade. In some embodiments, the method further comprises removing the scaffolds from the culture. In some embodiments, the method further comprises removing the scaffolds from the tissue. Scaffolds can be removed by any method known in the art, including for example by centrifugation.

Methods of Culturing with Recycled Vesicles

By another aspect, there is provided a method of culturing a first cell in media, the method comprising culturing in media comprising reduced growth factor concentration and comprising vesicles from a second cell, thereby culturing a first cell.

By another aspect, there is provided a method of culturing a first cell, the method comprising:
a. culturing the first cell in media for a time sufficient for the first cell to produce vesicles;
b. removing a portion of the media;
c. concentrating, purifying, or isolating the vesicles from the removed portion to produce a concentrated fraction;
d. returning the concentrated fraction to the culture comprising the first cell; and
e. continuing to culture the first cell;
   thereby culturing a first cell.

In some embodiments, culturing comprises increasing proliferation. In some embodiments, culturing comprises decreasing senescence. In some embodiments, the increasing/decreasing is as compared to cell cultured in standard conditions. In some embodiments, standard conditions are conditions without supplementation with vesicles. In some embodiments, standard conditions are culturing with growth factors. Methods of measuring proliferation and senescence are well known in the art and any such method may be employed. For example, cell counting or XTT assay may be used for measuring proliferation and activity of SA-beta-gal may be measured to assess senescence.

In some embodiments, increasing comprises an increase of at least 10, 20, 30, 40, 50, 60, 70, 80, 90, 100, 125, 150, 175, 200, 250, 300, 350, 400, 450, or 500 percent. Each possibility represents a separate embodiment of the invention. In some embodiments, increasing comprises an increase of at least 100%. In some embodiments, an increase is at least a doubling. In some embodiments, decreasing comprises a decrease of at least 10, 20, 30, 40, 50, 60, 70, 80, 90, 92, 95, 97, 99 or 100 percent. Each possibility represents a separate embodiment of the invention. In some embodiments, decreasing comprises a decrease of at least 50%. In some embodiments, decreasing comprises a decrease of at least 40%.

In some embodiments, the first cell is an MSC. In some embodiments, the first cell is a non-MSC. In some embodiments, the first cell is a primary cell. In some embodiments, the first cell is a stem cell. In some embodiments, the first cell is a terminally differentiated cell. In some embodiments, the first cell is a cell of a cell line. In some embodiments, the first cell is immortalized. In some embodiments, the first cell is not immortalized.

In some embodiments, the second cell is an MSC. In some embodiments, the second cell is not an MSC. In some embodiments, the second cell is a stem cell. In some embodiments, the second cell is a terminally differentiated cell. In some embodiments, the second cell is a cell of a cell line. In some embodiments, the second cell is immortalized. In some embodiments, the second cell is not immortalized.

In some embodiments, the media does not comprise serum. In some embodiments, the media does not comprise human platelet lysate. In some embodiments, the media is growth media. In some embodiments, the media is the growth media of the first cell. In some embodiments, the media comprises reduced growth factors. In some embodiments, continuing to culture is culturing with reduced growth factors. In some embodiments, in step (a) the first cell is grown with standard growth factors and in step (e) the first cell is grown with reduced growth factors. In some embodiments, the culturing in step (a) comprises reduced growth factors. In some embodiments, reduced growth factors are reduced growth factor concentration. In some embodiments, reduced is as compared to the growth factor concentration required to culture the first cell in the absence of the vesicles from the second cell. In some embodiments, reduced is as compared to the culture conditions in step (a). In some embodiments, the reduced is as compared to the standard growth conditions for the first cell. Standard growth conditions are well known in the art for various cell types and can be found on the websites of purveyors of various cells (for example at ATCC) and the websites of purveyors of culture media (for example Sigma Aldrich or Thermo Fisher). In some embodiments, the culturing in step (a) is in standard conditions. In some embodiments, reduced growth factors are devoid of growth factors.

In some embodiments, the culturing is in a vessel. In some embodiments, the media is in a vessel. In some embodiments, the vessel is a bioreactor. In some embodiments, the method further comprises adding the vesicles to the media. In some embodiments, the method further comprises adding the vesicles to the vessel. In some embodiments, the method further comprises contacting the media with the vesicles. In some embodiments, the vesicles comprise or consist of EVs from the second cell. In some embodiments, the vesicles comprise or consist of MBVs from the second cell. In some embodiments, the vesicles comprise or consist of both EVs and MBVs from the second cell.

In some embodiments, the portion consists of media. In some embodiments, the portion comprises ECM. In some embodiments, the portion comprises media and ECM. In some embodiments, the portion is at least 5, 10, 15, 20, 25, 30, 35, 40, 45, 50, 55, 60, 65, 70, 75, 80, 85 or 90% of the media. Each possibility represents a separate embodiment of the invention. In some embodiments, the portion is at most 10, 15, 20, 25, 30, 35, 40, 45, 50, 55, 60, 65, 70, 75, 80, 85, 90, 95, 97 or 99% of the media. Each possibility represents a separate embodiment of the invention. In some embodiments, the portion is between 10-90, 10-80, 10-75, 10-70, 10-60, 10-50, 10-40, 20-90, 20-80, 20-75, 20-70, 20-60, 20-50, 20-40, 25-90, 25-80, 25-75, 25-70, 25-60, 25-50, 25-40, 30-90, 30-80, 30-75, 30-70, 30-60, 30-50, 30-40, 40-90, 40-80, 40-75, 40-70, 40-60, 40-50, 50-90, 50-80, 50-75, 50-70, or 50-60% of the media. Each possibility represents a separate embodiment of the invention. In some embodiments, the portion is between 50-60% of the media. In some embodiments, the portion is at least 50%. In some embodiments, the portion is about 50%. Each possibility represents a separate embodiment of the invention. In some embodiments, the portion is at least 90%. In some embodiments, the portion is about 90%. In some embodiments, the portion is at least 95%. In some embodiments, the portion is about 95%. In some embodiments, the portion is about 100% of the media.

In some embodiments, the vesicles comprise or consist of EVs. In some embodiments, the vesicles comprise or consist of MBVs. In some embodiments, the vesicles comprise or consist of EVs and MBVs. In some embodiments, the vesicles are concentrated. In some embodiments, the vesicles are purified. In some embodiments, the vesicles are isolated.

In some embodiments, concentrating comprises removing at least 50, 60, 70, 75, 80, 90 or 95% of the liquid in the portion. Each possibility represents a separate embodiment of the invention. In some embodiments, returning the concentrated fraction comprises adding new media. In some embodiments, concentrating comprises purifying or isolating the vesicles from a portion of media and adding the concentrated fraction to less new media that was in the removed portion. In some embodiments, returning comprises returning the concentrated fraction with new media. In some embodiments, the new media is the same media as in step (a). In some embodiments, the new media comprises reduced growth factors. In some embodiments, the new media is devoid of growth factors.

In some embodiments, the first and second cell are the same cell type. In some embodiments, the first and second cell are from the same lineage. In some embodiments, the first and second cell are different cell types. In some embodiments, the first and second cells are from different lineages. In some embodiments, the first and second cells are from the same lineage but are different cell types.

In some embodiments, the first cell is an MSC and the method is a method of differentiating an MSC. In some embodiments, the second cell is a non-MSC. In some embodiments, the differentiating is toward a non-MSC cell fate.

In some embodiments, the method allows for a reduction of at least 10, 20, 25, 30, 40, 50, 60, 70, 75, 80, 90, 95, 97, 99 or 100% of the growth factors. Each possibility represents a separate embodiment of the invention. In some embodiments, reduced is at least 10, 20, 25, 30, 40, 50, 60, 70, 75, 80, 90, 95, 97, 99 or 100% reduced. Each possibility represents a separate embodiment of the invention.

By another aspect, there is provided a differentiated MSC produced by a method of the invention.

By another aspect, there is provide a tissue produced by a method of the invention.

By another aspect, there is provided an artificial tissue comprising at least two non-MSC cells derived from an MSC source.

In some embodiments, the MSC source is a single MSC source. In some embodiments, the MSC source is a single population of MSCs. In some embodiments, the MSC source is a single culturing of MSCs.

By another aspect, there is provided a composition comprising:
a. a population of cells;
b. media; and
c. isolated, purified or condensed vesicles.

In some embodiments, the composition comprises vesicles at a concentration higher than is found in nature. In some embodiments, the composition comprises vesicles at a concentration higher than is found by culturing the population of cells in the media. In some embodiments, the composition is an edible composition. In some embodiments, the composition is a therapeutic composition. In some embodiments, the composition is a graft.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

Cell sources: Muscle satellite cells are isolated from skeletal muscle biopsies. These can be obtained, for example from biopsies of live animals or animal carcasses. Satellite cells are identified by surface expression of CD56 and/or CD29 and absence of expression of CD45 and/or CD31. The cells are also positive or the transcription factor Pax7. These cells are expanded in culture and extracellular vesicles (EV) are then isolated.

Mesenchymal stromal cells (MSCs) are isolated from various tissues, including for example, adipose tissue, bone marrow, chorionic placenta, amniotic placenta, umbilical cord and dental pulp. The MSCs are grown in culture with or without serum. Cultures can be 2D, 3D, organoids and in bioreactors. The cells are expanded in culture and EVs/MBV can be isolated.

Myogenic differentiation: MSCs were incubated with at least one of the following compounds: TSA (50-200 nM), 5-Aza-2′-deoxycytidine (0.1-0.5 mM), IGF-1 (10-100 nM), the myostatin inhibitors Dorsomorphin (5 mM) and LDN193189 (0.5 mM). In addition, at least one of the following growth factors is added: BDNF, noggin, HGF, PDGF, FGF2 or IGF (10-100 ng/ml). Additionally, combination of a TGF-beta 1 receptor inhibitor (SB431542-2.5 mM), Wnt activator (BIO, 0.5 mM), and Shh inhibitor (erismodegib-10 mM) or each one of these compounds alone may be used.

Adipogenic differentiation of MSCs: MSCs are incubated with at least one of the following factors: adipocyte-derived EVs alone, adipocyte-derived EVs with insulin, 5 mg/ml, IGF-1, 1 mM ascorbat-2-phosphate, 500 nM dexamethasone, hydrocortisone 10 nM, 500 μM isobutyl-methylxanthine, 50 μM indomethacin, 1 μM rosiglitazone, 1 μM pioglitazone or 2 nM triiodothyronine. Alternative methods involve contacting the MSCs with at least one of cAMP, BMP4, BMP7, activators of Sirt6 and Sirt7, Sirt1 inhibitors, miR-124, miR-146, miR-30a, miR-30d, and miR-143.

Alternatively, MSCs are transferred to medium containing 0.25 UM insulin, 0.5 mM 1-methyl-3-isobutylxanthine (IBMX), 0.20 M dexamethasone, 2% FBS, and 15 μg/ml transferrin (Sigma) to induce differentiation. After 4 days of differentiation, the medium is changed to the same medium but without IBMX.

Alternatively, MSCs are incubated with EVs isolated from adipocytes or MSCs that were differentiated into adipose cells. These EVs are administered alone or with at least one of the above listed factors. Similarly, MSCs are plated with MBVs secreted by adipocytes or MSCs differentiated into adipose cells. The cells are expanded in culture and extracellular vesicles (EV) and MBVs are isolated. Alternatively, MBVs from fibroblasts are used.

EV isolated: EVs can be isolated by ultracentrifugation, by hollow fiber or tangential flow filtration. Other techniques known include column separation, filtration or centrifugation. The EVs may be further separated based on size, cargo (metabolomics, proteomics, total and small RNA seq) and surface markers. Their effects are screened as to their capacity to induce differentiation and as growth factors. Homogenous and well-defined particle populations are identified. EVs can also be isolated using the tissue culture ExoQuick kit.

Hereinbelow, EVs were prepared from cells, milk or plants using the ExoQuick-TC Ultra kit (SBI, Palo Alto, CA) according to the manufacturer's protocol. The protein content of the EVs was determined using Micro BCA assay kit and the expression of EV markers CD63 and CD81 was analyzed by Western blot.

MBV isolation: MBV are isolated from enzymatically digested ECM isolated from cultured cells. The ECM was collected as follows. Cells were cultured for 14 days and the media was removed. EVs and other soluble vesicles can then be isolated from the media (see above). The cells are then lysed using a gentle lysing solution (0.5% Triton in 20 mM ammonium hydroxide) for 5 min. The lysing solution does not damage the ECM. The plates are washed three times with HBSS including both Ca and Mg and then once with ultrapure water. The MBVs are isolated from the decellulized ECM by digestion with Liberase DL in Tris buffer with CaCl₂) (5 mM) and MgCl₂ (150 mM) for 1 hr. The isolated MVBs are centrifuged for 3 cycles at 500 g for 15 min, one cycle at 2500 g for an additional 15 min, and at 10,000 g for 30 min to remove the collagen fibril remnants. At the end of the process the MBVs are passed through a filter of 0.22 μm.

### Example 1: MSC Myogenic Differentiation by EVs/MBVs from Primary Muscle Cells

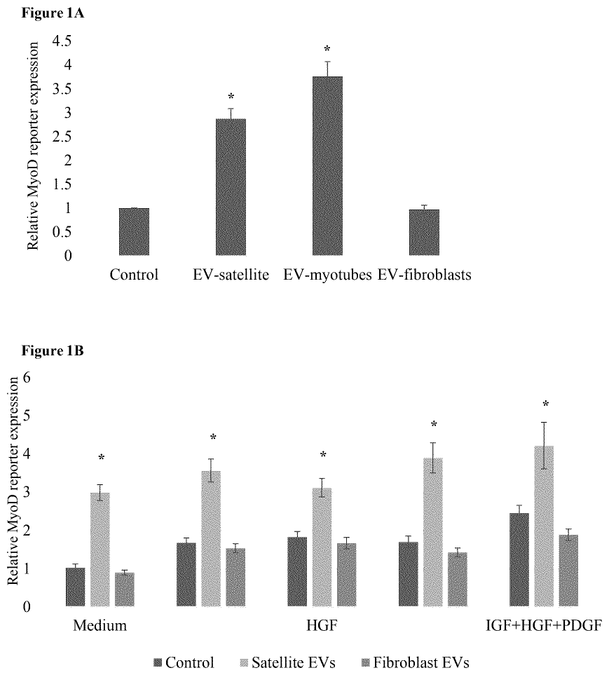

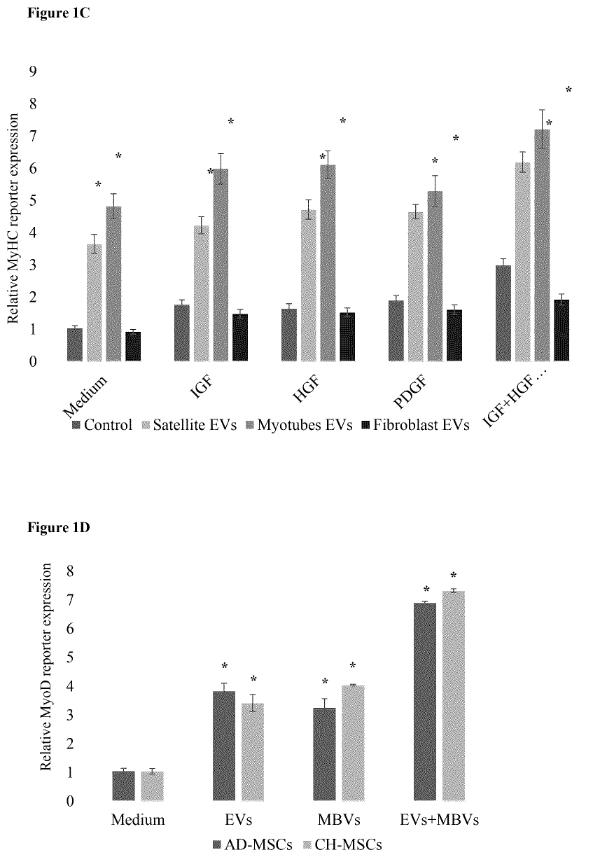

The effects of muscle cell derived extracellular vesicles (EVs) on the myogenic differentiation of MSCs was analyzed using a myoD reporter tagged to luciferase. Human satellite cells, myotubes and fibroblasts were grown in culture and EVs were isolated from each. MSCs were isolated from adipose tissue (AD-MSCs) and were transfected with the MyoD reporter. The cells grown in culture were treated with EVs (10\hat{ }8/ml) for 5 days and the luciferase activity was determined. As can be seen in FIG. 1A, both satellite cell and myotube-derived EVs induced an upregulation of the MyoD luciferase activity, whereas no significant effect was observed with EVs isolated from fibroblasts. Thus, the vesicles from these two cell types are effective at inducing myogenic differentiation.

Next, EVs were compared to the use of growth factors and the effect of combining EVs with growth factors on myogenic differentiation was tested. The same MyoD reporter was employed and MSCs were cultured with 10\hat{ }8 EVs (from satellite cells or fibroblasts) per ml of culture media. A no EV control was also performed. The EVs were also added in the presence of three different growth factors, IGF, HGF and PDGF or in the combined presence of all three growth factors. The no EV control was also performed with each growth factor and the triple combination. Each growth factor alone produced a very modest myogenic differentiation with less than a doubling of reporter activity over the untreated control (FIG. 1B, first column of each group). The EVs alone were superior to each growth factor and even the triple combination. Each growth factor alone combined with EVs produced a modest increase in MyoD expression over EVs alone and the three combined produced an even greater effect (FIG. 1B). Thus, though growth factors are inferior to EVs, their addition can enhance the EV's effect. A myosin heavy chain (MyHC) reporter was also used to measure myogenic differentiation. This reporter produced similar results to MyoD although with an even more pronounced difference between EVs and growth factors. (FIG. 1C).

Finally, human primary myotubes were growth in culture and EVs and membrane-bound vesicles (MBVs) were harvested every 3 days. MBVs are known to carry distinct cargos as compared to EVs. The vesicles were added to the culture of MSCs (adipose derived, AD; or chorionic placenta derived, CH) at a concentration of 10\hat{ }8 per ml of culture and myogenic differentiation was again monitored with a MyoD reporter. As seen for satellite cells and myotubes, the EVs from primary myocytes produced a robust expression of MyoD (FIG. 1D). This was observed in both types of MSCs. A similar result was also observed for the MBVs (FIG. 1D). Surprisingly, when the EVs and MBVs were mixed (in a 1:1 ratio) an even greater effect was observed (FIG. 1D) suggesting that these two vesicle types may function by different mechanisms.

### Example 2: MSC Myogenic Differentiation by EVs/MBVs from Differentiated MSCs

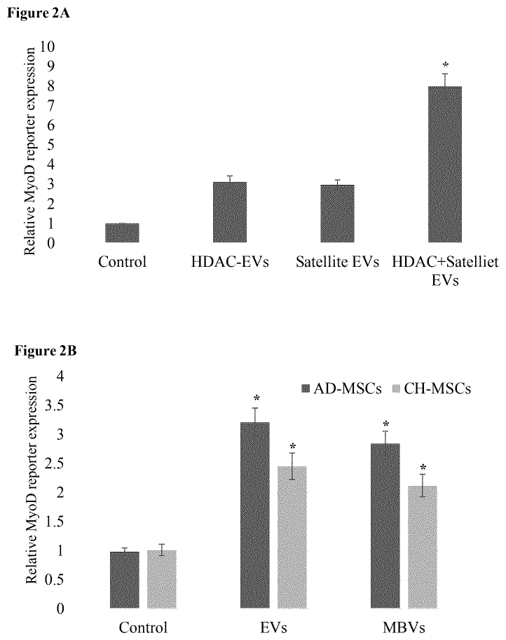

It was hypothesized that EVs from cells differentiated toward the muscle lineage could also induce myogenic differentiation. To test this, the effects of EVs secreted from MSCs treated with an HDAC inhibitor (and thus myogenically differentiated) were examined. AD-MSCs were treated with TSA (50 nm) for 24 hr. EVs were isolated and then new AD-MSCs were treated with these EVs alone, satellite cell-derived EVs alone or with a combination of these EVs. Although each of the EVs alone promoted myogenic differentiation (˜3-fold increase for both), surprisingly a combination of these EVs caused a synergistic effect with greatly increased MyoD reporter expression (8-fold increase) (FIG. 2A).

Next, MBVs from differentiated MSCs were examined. In this myogenic differentiation AD-MSCs were treated with a combination of 5-aza (0.5 μM), PDGF (10 ng/ml), BIO (0.5 μM) and SB431542 (2.5 μM) for 5 days, after which EVs and MBVs were isolated and administered to a new culture of AD-MSCs or CH-MSCs expressing the MyoD reporter. Not only did the EVs produce a robust increase in reporter expression, but it did so in both MSC types examined and the MBVs produced a similar result (FIG. 2B). As observed in primary cells, the combination of EVs and MBVs produced an even stronger combined effect, resulting in an increase of 6.9±0.643 in the reporter expression (P<0.01 as compared to individual effects of EVs or MBVs) once again showing an additive effect.

### Example 3: MSC Adipogenic Differentiation by EVs/MBVs from Primary Adipocytes

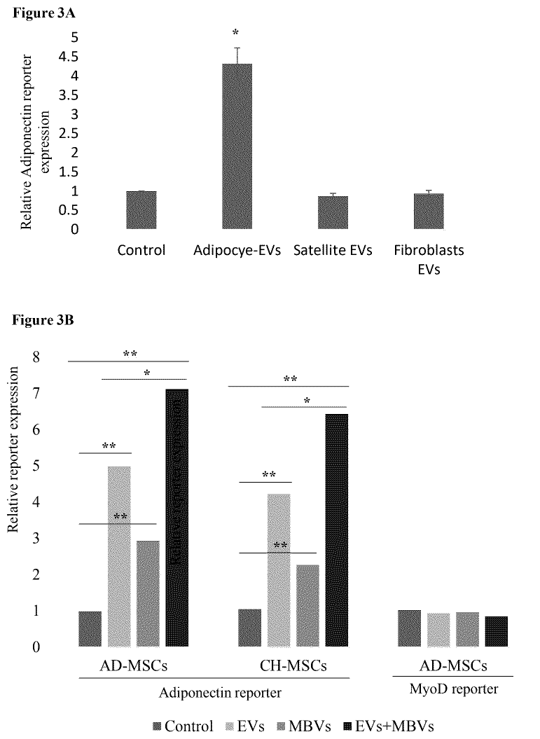

To promote adipogenic differentiation, EVs were isolated from primary adipocytes isolated from fat tissues. EVs from satellite cells and fibroblasts were also tested. The EVs were added to AD-MSCs for 5 days and the expression of an adiponectin reporter was measured. FIG. 3A shows that EVs isolated from adipocytes significantly induced upregulation of adiponectin in the AD-MSCs, whereas EVs from satellite cells and fibroblasts did not.

The ability of MBVs to induce adipogenic differentiation was also tested. Following three days in culture EV and MBVs were isolated from primary adipocytes and add to AD-MSC and CH-MSC culture. MBVs also enhanced differentiation, although not quite to the levels of EVs (FIG. 3B). As was observed for myogenic differentiation, the combination of EVs and MBVs produced an additive increase in reporter expression indicating that the two vesicles induce differentiation by different pathways (FIG. 3B). As a negative control, expression from a MyoD reporter in AD-MSCs was also checked, but as expected no increase in activity was produced by the EVs or MBVs from adipocytes (FIG. 3B).

Similar to what was performed for MSCs differentiated to a muscle cell fate, MSCs are differentiated to an adipose cell fate and their EVs and MBVs are tested. Adipogenic differentiation of MSCs is induced in the following media: DMEM+10% EV depleted serum, or chemically defined medium, supplemented with 500 nM Insulin, 100 nM dexamethasone, indomethacin and isobutyl-methyl-xanthine. EVs and MBVs are harvested from the differentiated MSCs at day 3 and added to AD-MSC or CH-MSCs. The adiponectin reporter is used to monitor adipogenic differentiation. EVs and MBVs from adipose differentiated MSCs are also able to induce adipogenic differentiation. Further, the combination of EVs and MBVs has an additive effect.

### Example 4: MSC Osteogenic Differentiation by EVs/MBVs

To promote osteogenic differentiation, EVs and MBVs were isolated from primary osteoblasts grown in culture for 3 days. The EVs and MBVs, alone or in combination, were added to AD-MSCs and CH-MSCs for 5 days and the expression of an osteocalcin reporter was measured. FIG. 4 shows that both EVs and MBVs isolated from osteoblasts significantly induced upregulation of osteocalcin in the MSCs, indicating osteogenic differentiation. Further, as observed for myogenic differentiation, the combination of EVs and MBVs produced an additive effect indicate they operate by different mechanisms.

Similar to what was performed for MSCs differentiated to a muscle cell fate, MSCs are differentiated to a bone cell fate. Osteogenic differentiation of MSCs is induced by culture with 10% EV depleted FBS, 0.01 microM dexamethasone, 50 microgram ascorbic acid, and 10 mM sodium-glycerophosphate. EVs and MBVs are harvested from the differentiated MSCs at day 3 and added to AD-MSC or CH-MSCs. The osteocalcin reporter is used to monitor osteogenic differentiation. EVs and MBVs from bone differentiated MSCs are also able to induce osteogenic differentiation. Further, the combination of EVs and MBVs has an additive effect.

### Example 5: EVs and MBVs from Other Mammals Show Similar Results

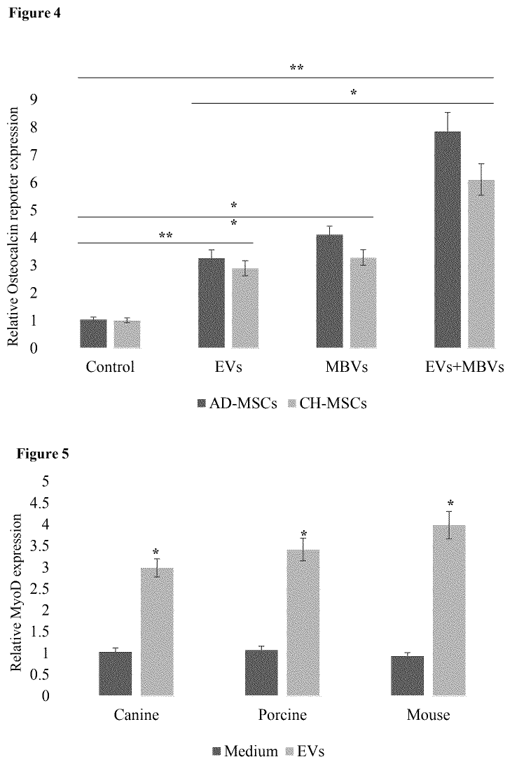

Skeletal muscle cells were harvested from mice, dogs and pigs and grown in culture. After 3 days EVs were harvested and administered to AD-MSCs from the same species. After 5 days myogenic differentiation was monitored by MyoD expression as assessed by RT-PCR. As can be seem in FIG. 5, EVs from these three species behave just as EVs from human MSCs do, inducing differentiation toward the same cell fate as the cells that produced the EVs.

### Example 6: Alternative EV Sources and MBV Isolation

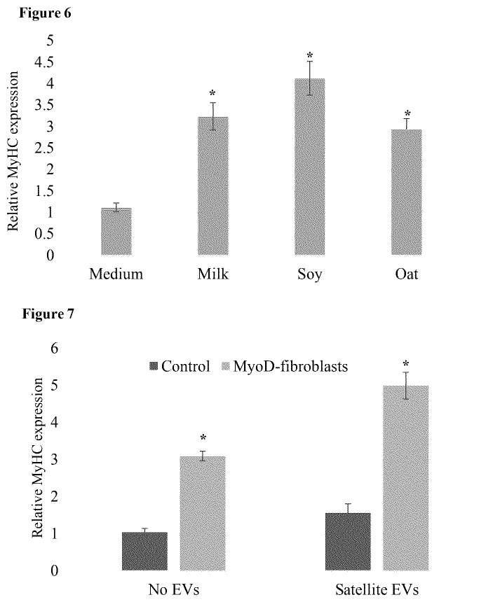

EVs secreted in milk and produced by various plants were assayed for their ability to promote muscle differentiation of MSCs. EVs were isolated by differential centrifugation and human AD-MSCs were cultured in the presence of the EVs for 5 days. Myogenic differentiation was monitored by MyHC expression as assessed by RT-PCR. As can be seem in FIG. 6, EVs from these three sources behave just as EVs from muscle cells do, inducing differentiation toward a muscle cell fate. This result is highly surprising as the source of the EVs is not muscle or related to muscle.

### Example 7: Transdifferentiation of Non-MSC Cells

Next it was tested if EVs could enhance the trans-differentiation of cells to a non-natural cell fate. Certain cells can be forced to differentiate to a cell fate that would not normally be possible in nature. Such forced differentiation is termed trans-differentiation. For example, fibroblasts can be forced to differentiated into a muscle cell fate by the ectopic expression of MyoD. To test the effect of EVs on trans-differentiation, EVs were isolated from human satellite cells as before. The EVs were added to a culture of normal fibroblasts and fibroblasts expressing MyoD. No EVs were added as a control. As can be seen in FIG. 7, MyoD expression in fibroblasts causes myogenic trans-differentiation as measured by MyHC expression. Satellite cell EVs produced a very modest, not statistically significant, increase as well. However, the combination of forced MyoD expression and EVs had a synergistic effect on trans-differentiation, with an increase in MyHC expression that was more than additive.

### Example 8: Optimized Culture and Scale Up Conditions

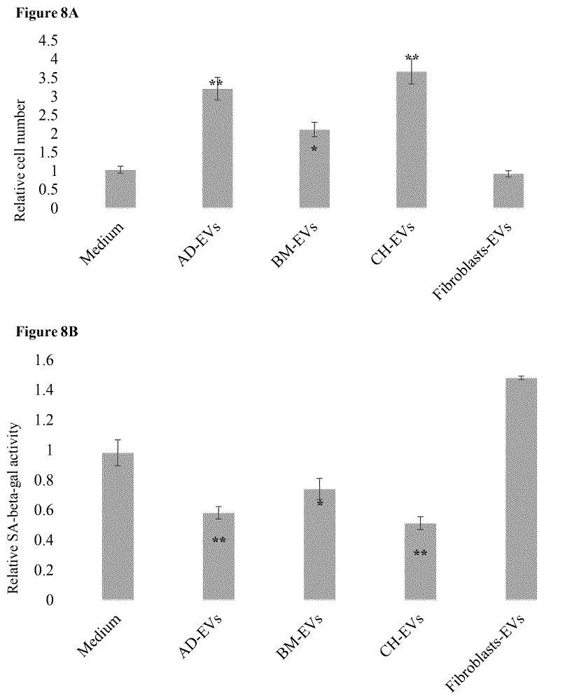

The ability of EVs to inhibit senescence and promote proliferation and thus their usefulness in replacing growth factors in tissue culture and for scale up for tissue culture was evaluated. EVs were isolated from AD-, bone marrow (BM-) and CH-MSCs grown in 1T75 flasks and administered to AD-MSCs growing in T225 flasks. Cell proliferation was monitored, and it was found that EVs from all of the MSCs produced a significant increase in proliferation as measured by relative cell number at a time point 5 days after the second passage of the cells (FIG. 8A). XTT cell proliferation assay kit was also used and produced comparable results. EVs from control fibroblasts did not produce any increase in proliferation. Activity of senescence associated beta-galactosidase activity (SA-beta-gal) was also measured in the MSCs cultured with EVs. EVs from all MSCs produced a significant decrease in SA-beta-gal activity, whereas EVs from fibroblasts actually produced a very slight increase (FIG. 8B). Interestingly, bone marrow MSCs produced the worst response by both measures, while CH-MSCs were slightly superior to AD-MSCs. This data taken together demonstrates that MSC EVs are a suitable supplement for tissue culture and can be used for scale up for large cell number growth in a bioreactor.

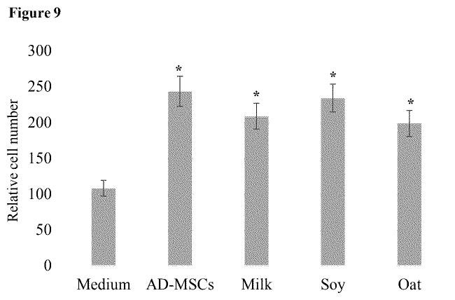

The ability of EVs from other sources to enhance proliferation was also tested. EVs were isolated from bovine milk, soy and oat and were administered to AD-MSCs for 5 days at which point proliferation was determined by comparing relative cell number. As can be seen in FIG. 9, the various EVs produced an increase in proliferation that was comparable to that of AD-MSCs. Thus, milk and plant EVs can also be used for supplementing tissue culture and for scale up.

EVs and MBVs are separated based on size, cargo (metabolomics, proteomics, and transcriptomics analysis) and surface markers. Moreover, they are separated based on their ability to induce differentiation and promote survival. The EVs and MBVs found to be most effective at promoting survival and differentiation to muscle and adipose are selected for further study. These selected EVs and MBVs are tested alone or in combination for their use in the scale up of tissue production in bioreactors. Their use is also compared to know growth conditions, including the use of growth factors in chemically defined media and serum (horse or fetal bovine). Complete or partial removal of these growth factors and replacement with EVs/MBVs is assayed for its effect on cell survival, differentiation and tissue formation.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of differentiating a mesenchymal stromal cell (MSC) to a first non-MSC cell fate, the method comprising contacting said MSC with purified or isolated extracellular vesicles (EVs), purified or isolated matrix-bound vesicles (MBVs) or a combination thereof derived from a second non-MSC source wherein said second non-MSC source is selected from a plant cell and from milk, thereby differentiating an MSC to a first non-MSC cell fate.

2. The method of claim 1, wherein said contacting comprises contacting with purified or isolated MBVs derived from a non-MSC source or MSC cell differentiated to a non-MSC fate or contacting with purified or isolated MBVs and purified or isolated EVs derived from a non-MSC source or MSC cell differentiated to a non-MSC fate.

3. The method of claim 1, wherein at least one of:
a. said MBVs are adhered to a microcarrier;
b. said MBVs are adhered to an artificial scaffold;
c. said isolated or purified EVs, MBVs or both are depleted of non-membrane enclosed growth factors;
d. said isolated or purified EVs, MBVs or both does not comprise conditioned media.

4. (canceled)

5. The method of claim 1, wherein said plant cell is a soy cell or an oat cell.

6. The method of claim 1, wherein at least one of:
a. said first and second non-MSC cell are from the same cell lineage;
b. said first and second non-MSC cell are the same cell type;
c. said first and second non-MSC cell are from a muscle lineage, and wherein said first and second non-MSC cells are selected from a satellite cell, a myoblast and a myotubule;
d. said first and second non-MSC cell are from an adipose lineage; and
e. said first and second non-MSC cell are from a bone lineage.

7. (canceled)

8. (canceled)

9. (canceled)

10. (canceled)

11. (canceled)

12. (canceled)

13. (canceled)

14. A method of producing artificial tissue, the method comprising:
a. culturing a population of MSCs in a vessel;
b. adding to said vessel a first set of vesicles comprising purified or isolated EVs, purified or isolated MBVs or both, wherein said first set of vesicles is capable of differentiating an MSCs to a first non-MSC cell fate; and
c. adding to said vessel a second set of vesicles comprising purified or isolated EVs, purified or isolated MBVs or both, wherein said second set of vesicles is capable of differentiating an MSCs to a second non-MSC cell fate;
wherein said first and second non-MSC cell fates are different cell fates, thereby producing artificial tissue.

15. The method of claim 14, wherein said first set of vesicles and said second set of vesicles are present in said vessel at the same time or said first set of vesicles and said second set of vesicles are added simultaneously.

16. (canceled)

17. The method of claim 14, wherein a first subpopulation of said population of MSCs differentiates to said first non-MSC cell fate and a second subpopulation of said population of MSCs differentiates to said second non-MSC cell fate.

18. The method of claim 14, further comprising culturing said population of MSCs with said first and second set of vesicles for a time sufficient for a first subpopulation of said population of MSCs to differentiate to said first non-MSC cell fate and a second subpopulation of said population of MSCs to differentiate to said second non-MSC cell fate.

19. The method of claim 14, wherein at least one of:
a. said first non-MSC cell fate and said second non-MSC cell fate are selected from muscle cells, adipose cells and bone cells; and
b. said artificial tissue is artificial meat and wherein said MSCs are non-human MSCs.

20. (canceled)

21. (canceled)

22. (canceled)

23. A method of culturing a first cell, the method comprising:
a. culturing said first cell in media for a time sufficient for said first cell to secrete vesicles;
b. removing a portion of said media;
c. concentrating, purifying or isolating secreted EVs, MBVs or both from said removed portion to produce a concentrated fraction; and
d. returning said concentrated fraction with new media to said culture comprising said first cell and continuing to culture said first cell;
thereby culturing a first cell.

24. The method of claim 23, wherein said continuing to culture said first cell comprises culturing with a reduced growth factor concentration as compared to a concentration of said growth factor required to culture said first cell in the absence of said extracellular vesicles.

25. (canceled)

26. The method of claim 24, wherein said second cell is a same cell type as said first cell.

27. The method of claim 24, wherein said second cell is a different cell type as said first cell.

28. The method of claim 23, wherein said first cell, said second cell or both is selected from a muscle cell, an adipose cell, a fibroblast, an MSC and an MSC differentiated toward a non-MSC cell fate.

29. (canceled)

30. The method of claim 23, wherein said first cell is an MSC and said method is a method of differentiating an MSC toward a non-MSC fate.

31. The method of claim 23, wherein said non-MSC fate is selected from a muscle cell fate and an adipocyte cell fate.

32. The method of claim 23, wherein at least one of:
a. said culturing is in a bioreactor; and
b. said media is chemically defined media.

33. (canceled)

34. The method of claim 23, wherein said isolated or purified EVs, MBVs or both comprises MBVs.

35. The method of claim 34, wherein said MBVs comprise a microcarrier coupled to said MBVs, an artificial scaffold coupled to said MBVs, a biodegradable microcarrier coupled to said MBVs or an edible microcarrier coupled to said MBVs.

36.-49. (canceled)
